(19) United States
(12) Patent Application Publication
(10) Pub. No.: US 2006/0062802 A1
(43) Pub. Date: Mar. 23, 2006

(54) Medicinal product

(76) Inventor: Peter Greifenstein, Raubling (DE)

Correspondence Address: PERRET DOISE;A PROFESSIONAL LAW CORPORATION, P.O. Box 3408, LAFAYETTE, LA, 70502-3408, US

(21) Appl. No.: 10/528,858

(22) PCT Filed: May 25, 2004
(86) PCT No.: PCT/DE04/01100

§ 371 Date: Mar. 23, 2005

(30) Foreign Application Priority Data
Apr. 13, 2004 (DE) 202004005959

(51) Int. Cl. A61K 36/899; A61K 36/86; A61K 36/736

(52) U.S. Cl. 424/195.15; 424/735; 424/750; 424/766; 424/777

(57) ABSTRACT

The present invention discloses a medicinal product with a content of ingredients and/or extracts of prunus armenica and of cocos nucifera and of humulus lupulus and germinated barley or germinated rye, or germinated wheat and of mycete and the liquid obtained from grape juice of grapevines through alcoholic fermentation, and from musaze and from rubus leaves, in each case as active ingredient, which is applicable to the treatment of AIDS, cancer or diseases of the psyche or nervous diseases.

The present invention relates to a phyto-pharmacological medicinal product for the treatment at least of Acquired Immune Deficiency Syndrome (AIDS) of cancer and diseases of the psyche or of the nervous system, having the characteristics given in the first portion of Claim 1.

From the state of the art a plurality of medicinal products are known for treatment of AIDS, cancer or neurological diseases, which on the one hand have considerable manufacturing costs and on the other hand are associated with a limited spectrum of effectiveness.

The object of the present invention is therefore to make available a medicinal product, which by the expenditure of extremely minor financial means can be easily and rapidly manufactured and has a very broad spectrum of effectiveness.

In accordance with the invention this object is solved in accordance with a type of medicinal product by means of the distinguishing part of the features given in Claim 1.

Particularly preferred embodiments are the subject of the dependent claims.

Within the framework of the present invention a medicinal product is provided, which comprises a content of ingredients and/or extracts of prunus armenica and of cocos nucifera and of humulus lupulus and germinated barley or germinated rye, or germinated wheat and of mycete, and the liquid obtained by alcoholic fermentation of the grape juice of grapevines, and from musaze and from rubus leaves, in each case as the active ingredient.

A particular advantage of these active components comprising medicinal products in accordance with the invention is that they are available on the world market and obtainable in large quantities at exceptionally favorable prices.

In accordance with the invention, the active ingredient components to be used can consist for example of their pulp, multiple fruit, juice, milk, kernels, fibers, cell filaments, myzelles, endosperm, leaves, blossoms, buds, hulls or stalks.

So, the medicinal product in accordance with the invention can for example have a content of pulp or kernels of prunus armenica and fibers or endosperm of cocos nucifera and multiple fruit of humulus lupulus and germinated barley or germinated oats or germinated rye or germinated wheat and cell filaments or myzelles of mycetes, and the liquid obtained by alcoholic fermentation of grape juice from the grapevine and the fruits or hulls of musaze and rubus leaves, in each case as an active ingredient.

In a preferred embodiment the medicinal product in accordance with the invention comprises in addition to the above ingredients, or extracts from the active ingredients, one or a plurality of common carrier materials, auxiliary means and/or additives and can the for example be made up in the form of a tablet or a sugar coated tablet or a suppository or drops.

As already indicated, the medicinal product can, instead of or in addition to the solid components of the active ingredient components, comprise one or a plurality of extracts from the plant active ingredients or active plant components.

This extract can be obtained for example by solid-liquid or liquid-liquid extraction of the individual components or the entire active ingredient component mixture with the help of common extraction means, and by subsequent partial or complete evaporation of the extraction solution.

In use of the concerned extraction, in accordance with the invention, it is a question of whether there is for example a hot or cold extraction as well as whether the extraction method is continuous or discontinuous.

Preferably, in accordance with the invention, we are dealing with a continuous extraction method by means of Soxhlet extraction, perforation or a percolation.

In the use of the discontinuous extraction in accordance with the invention it can be a matter of shaking out, leaching out or digestion.

In particularly preferred embodiments of the medicinal product in accordance with the invention the extract represents one of one fixed active ingredient content adjusted extract from one individual active ingredient, or from the entire mixture of active components.

This extract can for example be obtainable by means of maceration or percolation using ethanol or an ethanol-water mixture.

The extract which is to be used in accordance with the invention can consist of, for example, a dry extract (extracta sicca) and/or a liquid extract (extracta fluidica) and/or a viscous extract (extracta spissa)

The medicinal product in accordance with the invention can for example be compounded in the form of liquid to be taken in the form of drops or an aerosol or in the form of a solution for intravenous, intraarterial, intramuscular, subcutaneous or intralumbar injection or infusion.

In an especially preferred embodiment the medicinal product can have a content of ingredients and/or extracts of prunus armenica in the range for example of 10 wt % to 20 wt % and of cocus nucifera for example in the range from 10 wt % to 20 wt % and of humulus lupulus for example in the range from 10 wt % to 20 wt % and of germinated barley for example in the range from 10 to 20 wt % and of germinated rye in the range from 10 wt % to 20 wt % or germinated wheat from 10 wt % to 20 wt % and from mycete for example in the range from 10 wt % to 20 wt % and the liquid obtained by alcoholic fermentation of grape juice of the grapevine for example in the range from 10 wt % to 20 wt % and of musazes for example in the range from 10 wt % to 20 wt % and of rubus leaves for example in the range from 10 wt % to 20 wt %, in each case as the active ingredient and if necessary together with the usual carrier materials, auxiliary means and/or additive materials.

The ranges of weight percent data given above are presented above all for the reason that the active ingredient contents of the individual active material components vary case by case, at least somewhat depending on the condition of the growth substrate.

For the medicinal product in accordance with the invention, the active ingredients “mycete” can for example be chosen from the group, which includes chlorophyll-free, eukaryontic organisms, especially protoctista (fungus like protista) and/or fungi (higher fungi).

In particular the active ingredients in accordance with the invention “mycete” can be selected from the group as protoctista (lower fungi) myxomycote, myxomycete, acrasiomycete, plasmodiophoromycote, labyrinthulomycote oomycote, hypochytriomycote and chytridiomycote and as fungi (higher fungi) zygomycote, ascomycete, endomycete, ascomycete, basidiomycota, ustomycete, basidiomycete and includes imperfect fungi (deutermycete).

As “mycete” active ingredients components or extracts of dermatophyte, yeast or mildew can also be considered in accordance with the invention.

As active ingredients “the liquid obtained by alcoholic fermentation of the grape juice from grapevines” can, for example, be considered as wine, preferably white wine or red wine, especially from the Trollinger grape or Edelvernatsch.

Investigations have shown that the medicinal product in accordance with the invention is effective in both solid and liquid form for treatment among other things of Acquired Immune Deficiency Syndrome (AIDS) and/or is suitable for cancer, malignant tumors, carcinomas, sarcomas and/or diseases of the psyche or nervous system.

In accordance with the invention the components and/or extracts of prunus armenica and of cocos nucifera and of humulus lupulus and germinated barley or germinated rye, or germinated wheat and of mycete, and the liquid obtained from alcoholic fermentation of the grape juice of grapevines, and from musaze and from rubus leaves, for preparation of a medicinal product are useful for treatment of Acquired Immune Deficiency Syndrome (AIDS)

Alternatively or additionally the components and/or extracts of prunus armenica and of cocos nucifera and of humulus lupulus and germinated barley or germinated rye, or germinated wheat and of mycete and the liquid obtained from alcoholic fermentation of the grape juice of grapevines, and from musaze, and from rubus leaves for preparation of a medicinal product are useful for treatment of cancer, malignant tumors, carcinomas and sarcomas.

Alternatively or additionally the components and/or extracts content of prunus armenica and of cocos nucifera and of humulus lupulus and germinated barley or germinated rye, or germinated wheat and of mycete and the liquid obtained from alcoholic fermentation of the grape juice of grapevines, and from musaze, and from rubus leaves are useful for preparation of a medicinal product for treatment of diseases of the psyche or of the nervous system.

It has been shown furthermore that the components and/or extracts content of prunus armenica and of cocos nucifera and of humulus lupulus and germinated barley or germinated rye, or germinated wheat and of mycete and the liquid obtained from alcoholic fermentation of the grape juice of grapevines, and from musaze, and from rubus leaves for preparation of a medicinal product, are useful for treatment of diabetes diseases.

In summary it should be noted that the present invention, based on the use of especially cost effective active ingredients-components, and based on the manufacturability in accordance with the invention of the medicinal product by means of simple manufacturing methods, permits for the first time a cost effective, simple and rapid preparation of a medicinal product having a very broad spectrum of effectiveness for the treatment at least of AIDS, cancer and diseases of the psyche.

1. A medicinal product characterized in that through a content of components and/or extracts of prunus armenica and of cocos nucifera and of humulus lupulus and germinated barley or germinated rye, or germinated wheat and of mycete, and the liquid obtained from alcoholic fermentation of the grape juice of grapevines, and from musaze and from rubus leaves, in each case as active ingredient.

2. The medicinal product according to claim 1, characterized in that it consists of the active ingredient components in accordance with claim 1 and their pulp, multiple fruit, juice, milk, kernels, fibers, cell filaments, myzelles, endosperm, leaves, blossoms, buds, hulls or stalks.

3. The medicinal product according to claim 1, characterized in that through a content of pulp or kernels of prunus armenica and fibers or endosperm of cocos nucifera and multiple fruit of humulus lupulus and germinated barley or germinated oats or germinated rye or germinated wheat and cell filaments or myzelles of mycetes, and the liquid obtained by alcoholic fermentation of grape juice from the grapevine and the fruits or hulls of musaze and rubus leaves, in each case as an active ingredient.

4. The medicinal product according to claim 1, characterized in that in addition to the extracts and/or component extracts in accordance with claim 1 it comprises common carrier materials, auxiliary means and/or additives and is made up in the form of a tablet or a sugar coated tablet or a suppository or drops.

5. The medicinal product according to claim 1, characterized in that the extract can be obtained by solid-liquid or liquid-liquid extraction of the individual components or the active ingredient component mixture with the help of common extraction means, and by subsequent partial or complete evaporation of the extraction solution.

6. The medicinal product according to claim 1, characterized in that it is a question of whether there is hot or cold extraction as well as whether the extraction method is continuous or discontinuous.

7. The medicinal product according to claim 1, characterized in that the continuous extraction method is a Soxhlet extraction, perforation or a percolation, while the discontinuous extraction method can be a shaking out, leaching out or digestion.

8. The medicinal product according to claim 1, characterized in that the extract represents one of one fixed active ingredient content adjusted extract from one individual active ingredient or from the entire mixture of active components, which can be obtainable by means of maceration or percolation using ethanol or an ethanol-water mixtures.

9. The medicinal product according to claim 1, characterized in that the medicinal product is a dry extract (extracta sicca) and/or a liquid extract (extracta fluidica) and/or a viscous extract (extracta spissa).

10. The medicinal product according to claim 1, characterized in that it can be compounded in the form of a liquid to be taken in the form of drops or an aerosol or in the form of a solution for intravenous, intraarterial, intramuscular, subcutaneous or intralumbar injection or infusion.

11. The medicinal product according to claim 1, characterized in that it can have a content of ingredients and/or extracts of prunus armenica in the range of 10 wt % to 20 wt % and of cocus nucifera in the range from 10 wt % to 20 wt % and of humulus lupulus in the range from 10 wt % to 20 wt % and of germinated barley in the range from 10 to 20 wt % and of germinated rye in the range from 10 wt % to 20 wt % or germinated wheat from 10 wt % to 20 wt % and from mycete in the range from 10 wt % to 20 wt % and the liquid obtained by alcoholic fermentation of grape juice of the grapevine for example in the range from 10 wt % to 20 wt % and of musazes for example in the range from 10 wt % to 20 wt % and of rubus leaves in the range from 10 wt % to 20 wt %, in each case as the active ingredient together with the usual carrier materials, auxiliary means and/or additives.

12. The medicinal product according to claim 1 characterized in that the active ingredients mycete is selected from the group, which comprises chlorophyll-free, eukaryontic organisms, especially protoctista (fungus like protista) and/or fungi (higher fungi).

13. The medicinal product according to claim 1 characterized in that it is suitable for treatment of Acquired Immune Deficiency Syndrome (AIDS) and/or cancer, malignant tumors, carcinomas, sarcomas and/or diseases of the psyche or of the nervous system.

14. The method of using components and/or extracts of prunus armenica and of cocos nucifera and of humulus lupulus and germinated barley or germinated rye, or germinated wheat and of mycete, and the liquid obtained from alcoholic fermentation of the grape juice of grapevines, and from musaze and from rubus leaves, for preparation of a medicinal product for treatment of Acquired Immune Deficiency Syndrome (AIDS).

15. The method of using components and/or extracts or prunus armenica and of cocos nucifera and of humulus lupulus and germinated barley or germinated rye, or germinated wheat and of mycete and the liquid obtained from alcoholic fermentation of the grape juice of grapevines, and from musaze, and from rubus leaves for preparation of a medicinal product for treatment of cancer, malignant tumors, carcinomas and sarcomas.

16. The method of using components and/or extracts of prunus armenica and of cocos nucifera and of humulus lupulus and germinated barley or germinated rye, or germinated wheat and of mycete and the liquid obtained from alcoholic fermentation of the grape juice of grapevines, and from musaze, and from rubus leaves for preparation of a medicinal product for treatment of diseases of the psyche or of the nervous system.
